(19) United States
(12) Patent Application Publication
(10) Pub. No.: US 2025/0268565 A1
(43) Pub. Date: Aug. 28, 2025

(54) ULTRASONIC ENDOSCOPE

(71) Applicant: FUJIFILM Corporation, Tokyo (JP)

(72) Inventors: Karin HATTORI, Ashigarakami-gun (JP); Yasuhiko MORIMOTO, Ashigarakami-gun (JP); Kazushi FURUKAWA, Ashigarakami-gun (JP); Shozo IYAMA, Ashigarakami-gun (JP)

(73) Assignee: FUJIFILM Corporation, Tokyo (JP)

(21) Appl. No.: 19/051,182

(22) Filed: Feb. 12, 2025

(30) Foreign Application Priority Data
Feb. 28, 2024 (JP) 2024-028444

(51) Int. Cl. A61B 8/12; A61B 8/00

(52) U.S. Cl. CPC A61B 8/12; A61B 8/56

(57) ABSTRACT

An ultrasonic endoscope includes a distal end part including an ultrasound transmission and reception section, the distal end part includes a cable accommodation portion that accommodates a cable connected to the ultrasound transmission and reception section, and a filler that fills a gap in the cable accommodation portion, the cable includes plural signal cables that are electrically connected to ultrasonic oscillators included in the ultrasound transmission and reception section, a first covering member that bundles and covers the plural signal cables, and a second covering member that covers the first covering member, the cable has, in order from a side of the ultrasound transmission and reception section, a first region, a second region and a third region, and the filler is in contact with at least the first region and the second region.

### CROSS-REFERENCE TO RELATED APPLICATIONS

The present application claims priority under 35 U.S.C. § 119 to Japanese Patent Application No. 2024-028444 filed on Feb. 28, 2024. The above application is hereby expressly incorporated by reference, in its entirety, into the present application.

### BACKGROUND OF THE INVENTION

### 1. Field of the Invention

The present invention relates to an ultrasonic endoscope.

### 2. Description of the Related Art

JP2023-129671A describes a convex type ultrasonic endoscope. A filler layer that fills an inner space between an exterior member and a backing material layer and that has a role of fixing a substrate, a non-coaxial cable, and various wiring portions is provided at a distal end part of the ultrasonic endoscope.

WO2018/003737A describes a radial type ultrasonic endoscope. At a distal end part of the ultrasonic endoscope, filling materials are provided in a space of a connection portion between a substrate attached to a side surface of a backing material layer and a plurality of coaxial cables, a gap between the plurality of coaxial cables, and a gap through which the plurality of coaxial cables pass.

WO2018/003232A describes a convex type ultrasonic endoscope. A filler layer that fills a gap around a plurality of coaxial cables is provided at a distal end part of the ultrasonic endoscope.

### SUMMARY OF THE INVENTION

An object of the present disclosure is to provide an ultrasonic endoscope with improved durability.

An ultrasonic endoscope according to one embodiment of the technology of the present disclosure comprises: a distal end part including an ultrasound transmission/reception section, in which the distal end part has a cable accommodation portion that accommodates a cable connected to the ultrasound transmission/reception section, and a filler that fills a gap in the cable accommodation portion, the cable has a plurality of signal cables that are electrically connected to ultrasonic oscillators included in the ultrasound transmission/reception section, a first covering member that bundles and covers the plurality of signal cables, and a second covering member that covers the first covering member, the cable includes, in order from an ultrasound transmission/reception section side, a first region in which the signal cable is exposed, a second region in which the first covering member is exposed, and a third region in which the second covering member is exposed, in the cable accommodation portion, and the filler is in contact with at least the first region and the second region.

According to the technology of the present disclosure, a durability can be improved.

### BRIEF DESCRIPTION OF THE DRAWINGS

FIG. 1 is a schematic configuration diagram showing an example of an ultrasonic examination system 10 using an ultrasonic endoscope 12 according to an embodiment of the technology of the present disclosure.

FIG. 2 is a partially enlarged plan view showing a distal end part 40 shown in FIG. 1 and a vicinity thereof.

FIG. 3 is a cross-sectional view taken along line III-III shown in FIG. 2, and is a longitudinal cross-sectional view of the distal end part 40 taken along a center line thereof in a longitudinal axis direction.

FIG. 4 is a cross-sectional view taken along line IV-IV shown in FIG. 3, and is a transverse cross-sectional view taken along a center line of an arc structure of an ultrasonic oscillator array 50 of an ultrasonic observation part 36 of the distal end part 40.

FIG. 5 is a schematic view showing a cross section perpendicular to an axis of a signal cable 110.

FIG. 6 is a schematic view showing a cross section perpendicular to an axis of a cable 100.

FIG. 7 is an enlarged view of a portion including a substrate 60 and the cable 100.

FIG. 8 is a view showing a position of a filler 80 with a part of the cross section shown in FIG. 3 omitted.

FIG. 9 is a schematic cross-sectional view taken along line A-A of FIG. 7.

FIG. 10 is a view showing a modification example of a protrusion 102A.

FIG. 11 is a view showing a modification example of the cable 100 and is a view corresponding to FIG. 7.

### DESCRIPTION OF THE PREFERRED EMBODIMENTS

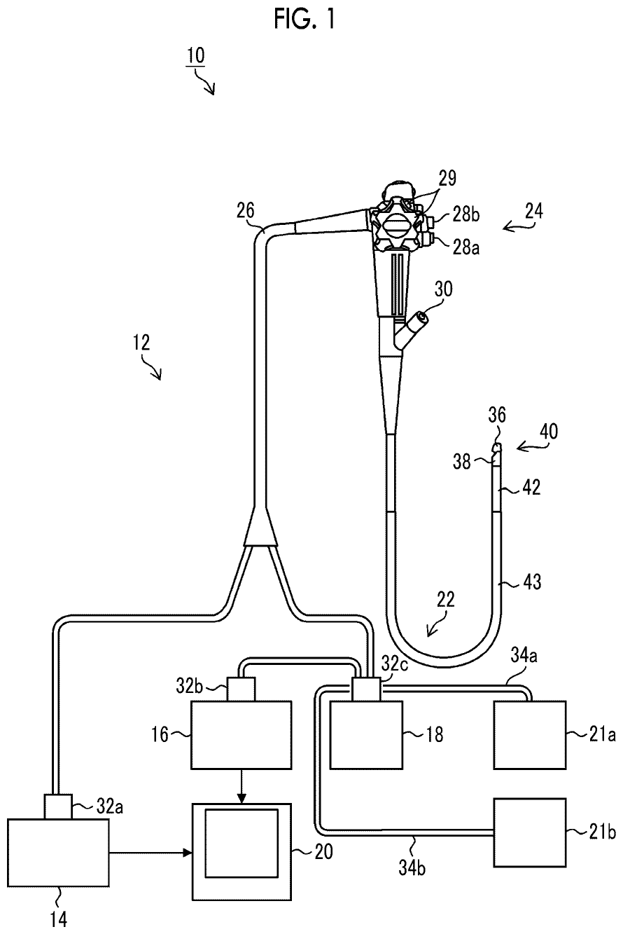

FIG. 1 is a schematic configuration diagram showing an example of an ultrasonic examination system 10 using an ultrasonic endoscope 12 according to an embodiment of the technology of the present disclosure. The ultrasonic examination system 10 comprises the ultrasonic endoscope 12, an ultrasound processor device 14 that generates an ultrasound image, an endoscope processor device 16 that generates an endoscopic image, a light source device 18 that supplies illumination light for illuminating an inside of a body cavity to the ultrasonic endoscope 12, a monitor 20 that displays the ultrasound image and the endoscopic image, a water supply tank 21a that stores cleaning water and the like, and a suction pump 21b that suctions an object to be suctioned in the body cavity.

The ultrasonic endoscope 12 has an insertion part 22 that is inserted into a body cavity of a subject, an operating part 24 that is consecutively provided in a proximal end part of the insertion part 22 and is used by an operator to perform an operation, and a universal cord 26 that has one end connected to the operating part 24.

In the operating part 24, an air/water supply button 28a that opens and closes an air/water supply pipe line (not shown) from the water supply tank 21a, and a suction button 28b that opens and closes a suction pipe line (not shown) from the suction pump 21b are provided side by side. In the operating part 24, a pair of angle knobs 29 and a treatment tool insertion port 30 are provided.

In the other end part of the universal cord 26, an ultrasound connector 32a that is connected to the ultrasound processor device 14, an endoscope connector 32b that is connected to the endoscope processor device 16, and a light source connector 32c that is connected to the light source device 18 are provided. The ultrasonic endoscope 12 is attachably and detachably connected to the ultrasound processor device 14, the endoscope processor device 16, and the light source device 18 via the connectors 32a, 32b, and 32c, respectively. The connector 32c comprises an air/water supply tube 34a that is connected to the water supply tank 21a, and a suction tube 34b that is connected to the suction pump 21b.

The insertion part 22 includes, in order from a distal end side, a distal end part 40 that has an ultrasonic observation part 36 and an endoscope observation part 38, a bendable part 42 that is consecutively provided on a proximal end side of the distal end part 40, and a soft part 43 that connects a proximal end side of the bendable part 42 and a distal end side of the operating part 24.

The bendable part 42 is remotely operated to be bent by rotationally operating the pair of angle knobs 29 provided in the operating part 24. With this, the distal end part 40 can be directed in a desired direction.

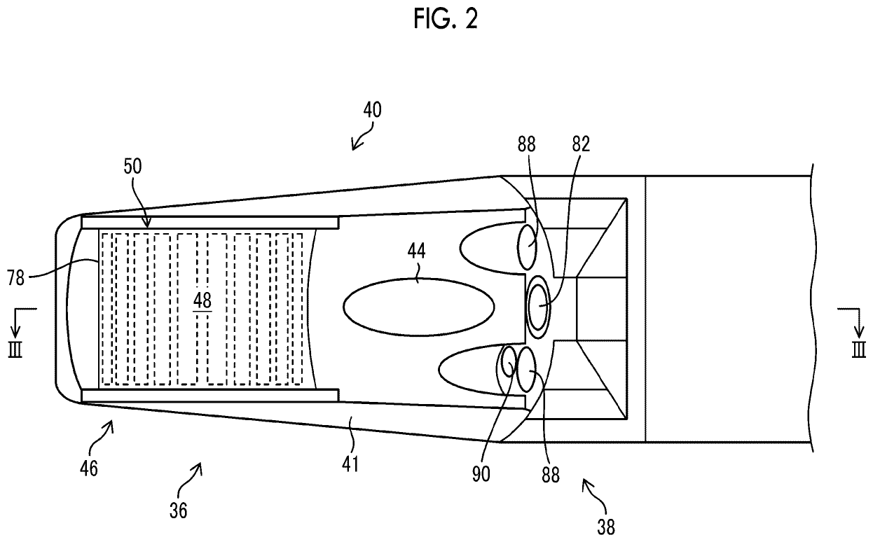

The ultrasound processor device 14 generates and supplies an ultrasound signal for causing an ultrasonic oscillator array 50 of an ultrasonic oscillator unit 46 (see FIG. 2) of the ultrasonic observation part 36 to generate ultrasonic waves. The ultrasound processor device 14 receives and acquires an echo signal reflected from an observation target site irradiated with the ultrasonic wave, by the ultrasonic oscillator array 50 and executes various kinds of signal processing on the acquired echo signal to generate an ultrasound image that is displayed on the monitor 20.

The endoscope processor device 16 receives and acquires a captured image signal acquired from the observation target site illuminated with illumination light from the light source device 18 in the endoscope observation part 38, and executes various kinds of processing on the acquired captured image signal to generate an endoscopic image that is displayed on the monitor 20.

In the example of FIG. 1, the ultrasound processor device 14 and the endoscope processor device 16 are configured with two devices (computers) provided separately. However, the present disclosure is not limited thereto, and both the ultrasound processor device 14 and the endoscope processor device 16 may be configured with one device.

In order to image an observation target site inside a body cavity using the endoscope observation part 38 to acquire a captured image signal, the light source device 18 generates illumination light, such as white light including light of three primary colors of red light, green light, and blue light or light of a specific wavelength. The illumination light propagates through a light guide (not shown) and the like in the ultrasonic endoscope 12 and is emitted from the endoscope observation part 38, and the observation target site inside the body cavity is illuminated with the illumination light.

The monitor 20 receives video signals generated by the ultrasound processor device 14 and the endoscope processor device 16 and displays the ultrasound image and the endoscopic image. In regard to the display of the ultrasound image and the endoscopic image, only one of these images may be appropriately switched and displayed on the monitor 20 or both the images may be displayed simultaneously.

In the present embodiment, although the ultrasound image and the endoscopic image are displayed on one monitor 20, a monitor for ultrasound image display and a monitor for endoscopic image display may be provided separately. In addition, the ultrasound image and the endoscopic image may be displayed in a display form other than the monitor 20, for example, in a form of being displayed on a display of a terminal carried by the operator.

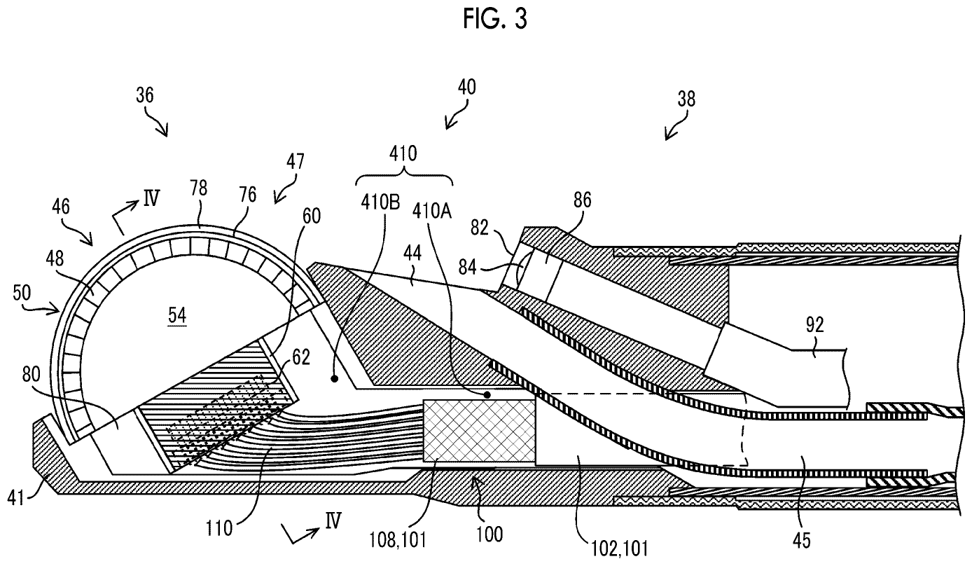

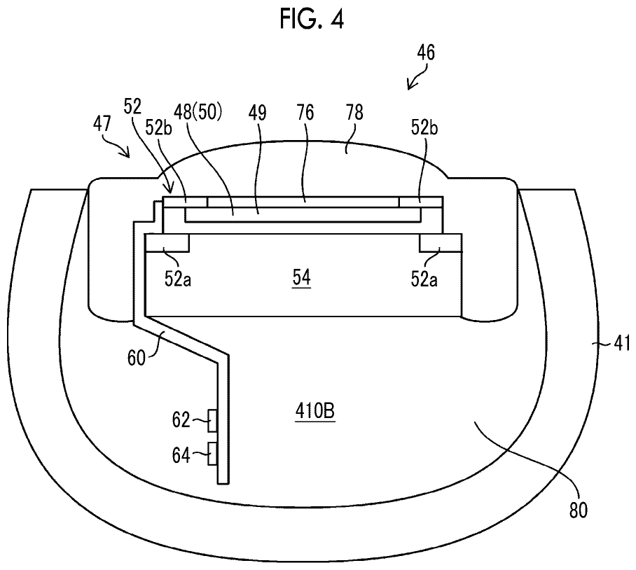

Next, a configuration of the distal end part 40 will be described with reference to FIGS. 2 to 4. FIG. 2 is a partially enlarged plan view showing the distal end part 40 shown in FIG. 1 and a vicinity thereof. FIG. 3 is a cross-sectional view taken along line III-III shown in FIG. 2, and is a longitudinal cross-sectional view of the distal end part 40 taken along a center line thereof in a longitudinal axis direction. FIG. 4 is a cross-sectional view taken along line IV-IV shown in FIG. 3, and is a transverse cross-sectional view taken along a center line of an arc structure of the ultrasonic oscillator array 50 of the ultrasonic observation part 36 of the distal end part 40.

As shown in FIGS. 2 and 3, in the distal end part 40, the ultrasonic observation part 36 for acquiring the ultrasound image is mounted on the distal end side, and the endoscope observation part 38 for acquiring the endoscopic image is mounted on the proximal end side. In the distal end part 40, a treatment tool outlet port 44 is provided between the ultrasonic observation part 36 and the endoscope observation part 38.

The endoscope observation part 38 comprises an observation window 82, an objective lens 84, an imaging element 86, an illumination window 88, a cleaning nozzle 90, a wiring cable 92, and the like. The observation window 82, the objective lens 84, the imaging element 86, and the illumination window 88 constitute an imaging unit.

The treatment tool outlet port 44 is connected to a treatment tool channel 45 that is inserted into the insertion part 22. A treatment tool (not shown) inserted from the treatment tool insertion port 30 of FIG. 1 is led out from the treatment tool outlet port 44 into the body cavity via the treatment tool channel 45.

As shown in FIGS. 2 to 4, the ultrasonic observation part 36 comprises the ultrasonic oscillator unit 46 constituting an ultrasound transmission/reception section, an exterior member 41 that holds the ultrasonic oscillator unit 46, and a cable 100 that is electrically connected to the ultrasonic oscillator unit 46 via a substrate 60. The cable 100 forms an elongated shape extending along a longitudinal axis direction of the insertion part 22, and is provided to extend to the connector 32a.

The exterior member 41 is made of a hard member, such as hard resin, and constitutes a part of the distal end part 40. In the exterior member 41, an accommodation space 410 that penetrates the exterior member 41 in the longitudinal axis direction of the insertion part 22 is provided. The accommodation space 410 includes a first space 410A on the proximal end side and a second space 410B on the distal end side, which is larger than the first space 410A. A part of the ultrasonic oscillator unit 46, the substrate 60, and a distal end side of the cable 100 are accommodated in the accommodation space 410. The accommodation space 410 constitutes a cable accommodation portion that accommodates the cable 100.

The ultrasonic oscillator unit 46 has the ultrasonic oscillator array 50 consisting of a plurality of the ultrasonic oscillators 48, an electrode 52 provided on an end part side of the ultrasonic oscillator array 50 in a width direction (direction perpendicular to the longitudinal axis direction of the insertion part 22), a backing material layer 54 that supports each ultrasonic oscillator 48 from a lower surface side, and the substrate 60 that is disposed along a side surface of the backing material layer 54 in the width direction and is connected to the electrode 52.

As long as the substrate 60 can electrically connect the plurality of ultrasonic oscillators 48 and the cable 100, in particular, a structure thereof is not limited.

It is preferable that the substrate 60 is configured with, for example, a wiring substrate, such as a flexible substrate (flexible printed substrate (also referred to as a flexible printed circuit (FPC)) having flexibility, a printed wiring circuit substrate (also referred to as a printed circuit board (PCB)) made of a rigid substrate having high rigidity with no flexibility, or a printed wiring substrate (also referred to as a printed wired board (PWB)).

The ultrasonic oscillator unit 46 has an acoustic matching layer 76 laminated on the ultrasonic oscillator array 50, and an acoustic lens 78 laminated on the acoustic matching layer 76. The ultrasonic oscillator unit 46 is configured as a laminate 47 having the acoustic lens 78, the acoustic matching layer 76, the ultrasonic oscillator array 50, and the backing material layer 54.

The ultrasonic oscillator array 50 is configured with a plurality of rectangular parallelepiped ultrasonic oscillators 48 arranged in a convex arc shape outward. The ultrasonic oscillator array 50 is, for example, an array of 48 channels to 192 channels consisting of 48 to 192 ultrasonic oscillators 48. Each ultrasonic oscillator 48 has a piezoelectric body 49.

The ultrasonic oscillator array 50 has the electrode 52. The electrode 52 has an individual electrode 52a individually and independently provided for each ultrasonic oscillator 48, and an oscillator ground 52b that is a common electrode common to all the ultrasonic oscillators 48. In FIG. 4, a plurality of the individual electrodes 52a are disposed on lower surfaces of end parts of the plurality of ultrasonic oscillators 48, and the oscillator ground 52b is disposed on upper surfaces of the end parts of the ultrasonic oscillators 48.

The substrate 60 has 48 to 192 wirings (not shown) that are electrically connected to the individual electrodes 52a of 48 to 192 ultrasonic oscillators 48, respectively, and a plurality of electrode pads 62 that are connected to the ultrasonic oscillators 48 through the wirings, respectively.

The ultrasonic oscillator array 50 has a configuration in which a plurality of the ultrasonic oscillators 48 are arranged at a predetermined pitch in a one-dimensional array shape as an example. The ultrasonic oscillators 48 constituting the ultrasonic oscillator array 50 are arranged at regular intervals in a convexly curved shape along the longitudinal axis direction of the insertion part 22, and are sequentially driven based on a driving signal input from the ultrasound processor device 14 (see FIG. 1). With this, convex electronic scanning is performed with a range in which the ultrasonic oscillators 48 shown in FIG. 2 are arranged, as a scanning range.

The acoustic matching layer 76 is provided for taking acoustic impedance matching between the subject and the ultrasonic oscillators 48.

The acoustic lens 78 is provided for converging the ultrasonic waves emitted from the ultrasonic oscillator array 50 toward the observation target site. The acoustic lens 78 is formed of, for example, a silicone-based resin (such as a millable silicone rubber or a liquid silicone rubber), a butadiene-based resin, a polyurethane-based resin, or the like. In the acoustic lens 78, powder, such as titanium oxide, alumina, or silica, is mixed as necessary. With this, the acoustic lens 78 can take acoustic impedance matching between the subject and the ultrasonic oscillators 48 in the acoustic matching layer 76, and can increase a transmittance of the ultrasonic waves.

As shown in FIGS. 3 and 4, the backing material layer 54 is disposed on an inner side with respect to an arrangement surface of a plurality of the ultrasonic oscillators 48, that is, a rear surface (lower surface) of the ultrasonic oscillator array 50. The backing material layer 54 is composed of a layer of a member made of a backing material. The backing material layer 54 has a role of mechanically and flexibly supporting the ultrasonic oscillator array 50 and attenuating ultrasonic waves propagated to the backing material layer 54 side among ultrasound signals oscillated from the plurality of ultrasonic oscillators 48 or reflected and propagated from an observation target. The backing material is made of a material having rigidity, such as hard rubber, and an ultrasonic wave attenuating material (ferrite, ceramics, or the like) is added as necessary.

The substrate 60 shown in FIG. 4 has a plurality of the electrode pads 62 that are electrically connected to the plurality of individual electrodes 52a at one end, and a ground electrode pad 64 that is electrically connected to the oscillator ground 52b. In FIG. 4, the cable 100 is omitted.

Electrical bonding of the substrate 60 and the individual electrodes 52a can be established by, for example, a resin material having conductivity. Examples of the resin material include an anisotropic conductive film (ACF) or an anisotropic conductive paste (ACP) obtained by mixing fine conductive particles with a thermosetting resin and forming the mixture into a film.

As another resin material, for example, a resin material in which conductive fillers, such as metallic particles, are dispersed into a binder resin, such as epoxy or urethane, and the fillers form a conductive path after adhesion may be used. Examples of this resin material include a conductive paste, such as a silver paste.

As shown in FIG. 3, the cable 100 includes a plurality of signal cables 110 and a tubular covering portion 101 that bundles and covers the plurality of signal cables 110.

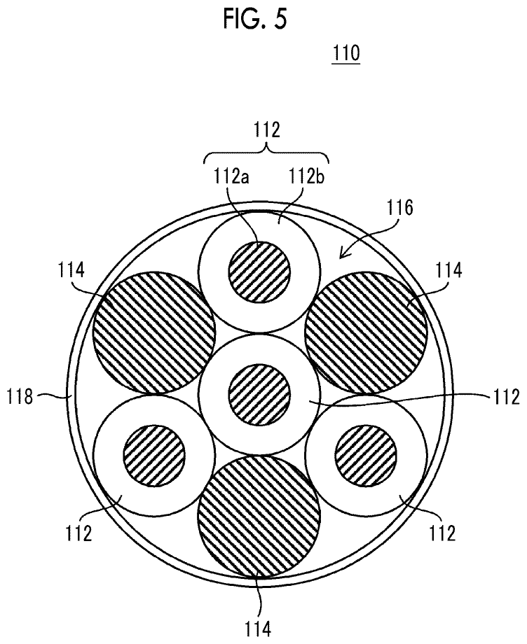

FIG. 5 is a schematic view showing a cross section perpendicular to an axis of the signal cable 110. In the example of FIG. 5, the signal cable 110 is a non-coaxial cable. The signal cable 110 includes a plurality of signal lines 112 and a plurality of ground lines 114. The signal line 112 is composed of, for example, a conductor 112a and an insulating layer 112b that covers an outer peripheral surface of the conductor 112a.

The conductor 112a is composed of, for example, an element wire of copper, a copper alloy, or the like. The element wire is subjected to, for example, a plating treatment, such as tin plating or silver plating. The conductor 112a has, for example, a diameter of 0.03 mm to 0.04 mm. The insulating layer 112b can be made of, for example, a resin material, such as fluorinated-ethylene-propylene (FEP) or perfluoroalkoxy (PFA). The insulating layer 112b has, for example, a thickness of 0.015 mm to 0.025 mm.

The ground line 114 is composed of, for example, a conductor having the same diameter as the signal line 112. The ground line 114 is composed of an element wire of copper or a copper alloy or a twisted wire obtained by twisting a plurality of element wires of copper or a copper alloy.

A first signal line bundle 116 is configured by twisting together a plurality of the signal lines 112 and a plurality of the ground lines 114.

The signal cable 110 comprises a sheath 118 of the first signal line bundle that bundles and covers the first signal line bundle 116. The sheath 118 of the first signal line bundle can be made of an insulating film obtained by laminating metal foils via an adhesive, or the like. The insulating film is made of a polyethylene terephthalate (PET) film or the like. The metal foil is made of an aluminum foil, a copper foil, or the like.

The signal cable 110 is shielded by the sheath 118 of the first signal line bundle with the plurality of signal lines 112 as one set.

The first signal line bundle 116 is configured by twisting seven lines including four signal lines 112 and three ground lines. One signal line 112 of the four signal lines 112 is disposed at a center. The remaining three signal lines 112 and three ground lines 114 are disposed adjacently in a periphery of the signal line 112 at the center. However, the number of signal lines 112, the number of ground lines 114, and arrangement thereof in the first signal line bundle 116 are not limited to a structure of FIG. 5. Each conductor 112a included in the signal cable 110 is electrically connected to any of the electrode pads 62 of the substrate 60.

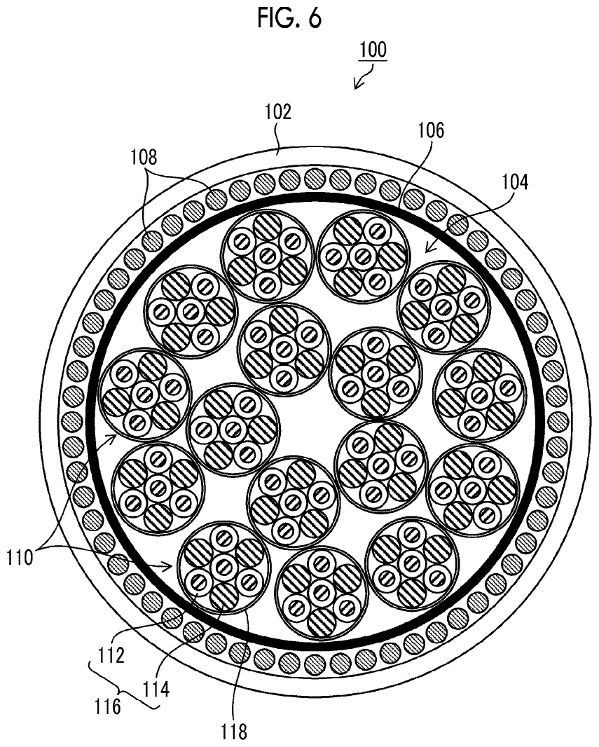

FIG. 6 is a schematic view showing a cross section perpendicular to an axis of the cable 100. In the example of FIG. 6, the cable 100 comprises a plurality of the signal cables 110, a tubular resin layer 106 that bundles and covers the plurality of signal cables 110, a tubular shield layer 108 that is provided along an outer peripheral surface of the resin layer 106 and covers the outer peripheral surface of the resin layer 106, and a tubular outer cover 102 that is provided along an outer peripheral surface of the shield layer 108 and covers the outer peripheral surface of the shield layer 108. The covering portion 101 is configured with the resin layer 106, the shield layer 108, and the outer cover 102.

The outer cover 102 can be made of a fluorine-based resin material, such as PFA, FEP, an ethylene-tetrafluoroethylene copolymer (ETFE), polyvinyl chloride (PVC), or the like, which is extrusion-molded. The outer cover 102 constitutes an outermost peripheral surface of the cable 100. It is preferable that an outer surface of the outer cover 102 has a high smoothness in order to reduce friction with other contents (an air/water supply tube, a suction tube, a pulling wire, or the like) inside the ultrasonic endoscope 12 and to improve robustness.

The resin layer 106 can be formed of, for example, the above-described fluorine-based resin material or a tape made of a resin.

It is preferable that an outer surface of the shield layer 108 has a smoothness lower than the smoothness of the outer surface of the outer cover 102. The smoothness can be defined by, for example, an average surface roughness. The shield layer 108 is, for example, a metal mesh shield formed by braiding a plurality of element wires. The element wire is made of a copper wire, a copper alloy wire, or the like subjected to a plating treatment (tin plating or silver plating). The resin layer 106 and the shield layer 108 constitute a first covering member that bundles and covers the plurality of signal cables 110. The resin layer 106 is not essential to the cable 100 and may be omitted. The outer cover 102 constitutes a second covering member that covers the first covering member.

The shield layer 108 is provided in a concentric circular shape with the resin layer 106 on an outer periphery of the resin layer 106, and surrounds and covers the outer peripheral surface of the resin layer 106 in a range of 360 degrees in a circumferential direction. The outer cover 102 is provided in a concentric circular shape with the shield layer 108 on an outer periphery of the shield layer 108, and surrounds and covers the outer peripheral surface of the shield layer 108 in a range of 360 degrees in a circumferential direction.

In the example of FIG. 6, the cable 100 includes 16 signal cables 110 and includes 64 signal lines 112. The number of signal cables 110 and the number of signal lines 112 are not limited to these numerical values.

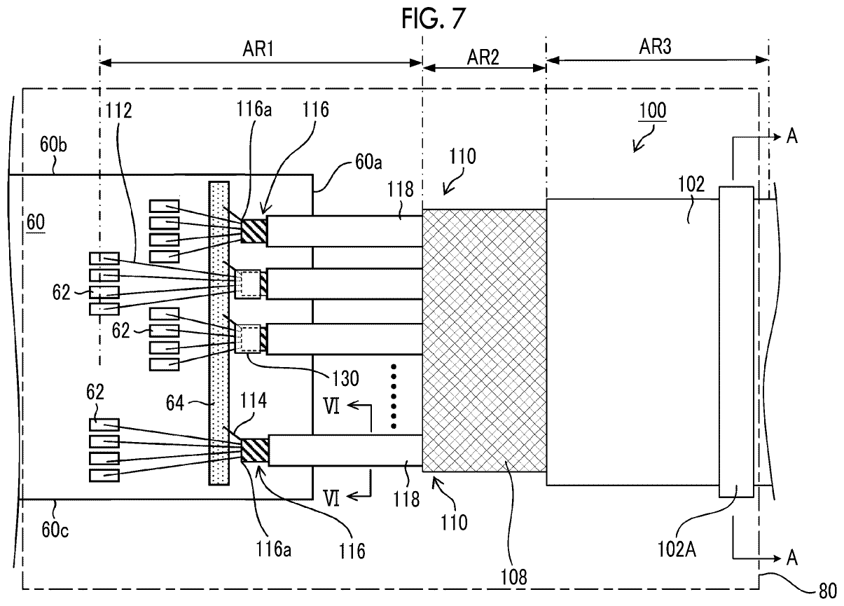

FIG. 7 is an enlarged view of a portion including the substrate 60 and the cable 100. As shown in FIG. 7, the substrate 60 has a plurality of the electrode pads 62 disposed along a side 60a on the proximal end side, and the ground electrode pad 64 disposed between the plurality of electrode pads 62 and the side 60a. The ground electrode pad 64 is disposed in parallel to the side 60a.

The cable 100 is disposed at a position facing the side 60a of the substrate 60. The electrode pad 62 and the signal line 112 of the signal cable 110 are electrically connected to each other. The signal cable 110 is disposed in parallel to a side 60b and a side 60c that are perpendicular to the side 60a. However, a positional relationship between the substrate 60 and the signal cable 110 is not particularly limited.

As shown in FIGS. 3 and 7, the cable 100 has a first region AR1 in which the covering portion 101 is peeled off and the signal cable 110 is partially exposed, on the distal end side thereof. The cable 100 has a second region AR2 in which the outer cover 102 is peeled off and the shield layer 108 is partially exposed, on the proximal end side with respect to the first region AR1. The cable 100 has a third region AR3 in which the outer cover 102 is exposed, on the proximal end side with respect to the second region AR2. As described above, in the accommodation space 410, the cable 100 comprises, in order from the ultrasonic oscillator unit 46 side, the first region AR1 in which the signal cable 110 is exposed, the second region AR2 in which the shield layer 108 is exposed, and the third region AR3 in which the outer cover 102 is exposed.

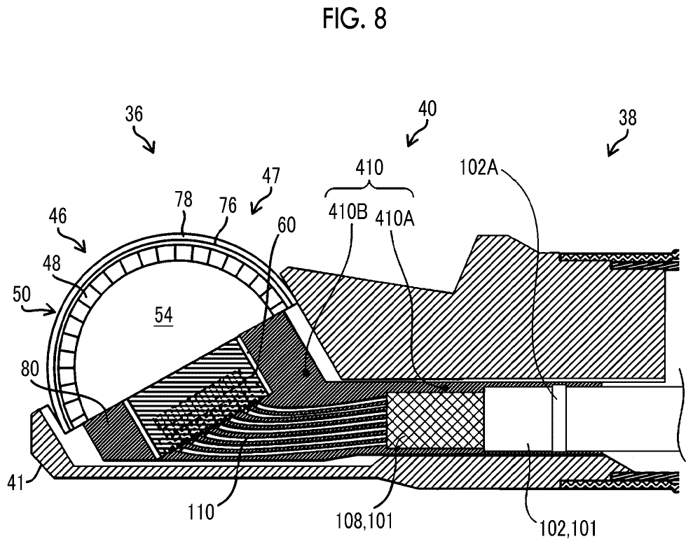

In the accommodation space 410 shown in FIG. 3, in a gap between the exterior member 41 and the ultrasonic oscillator unit 46, the substrate 60, and the distal end side of the cable 100 (a portion other than the cable 100 in the first space 410A and a portion other than the ultrasonic oscillator unit 46, the substrate 60, and the cable 100 in the second space 410B), a filler 80 is provided to fill the gap. FIG. 8 is a view showing a position of the filler 80 with a part of the cross section shown in FIG. 3 omitted.

The filler 80 mainly has a role of fixing the substrate 60, the signal cable 110, and various wiring portions. It is preferable that an acoustic impedance of the filler 80 matches an acoustic impedance of the backing material layer 54 with a certain accuracy or higher such that the ultrasound signals propagated from the ultrasonic oscillator array 50 to the backing material layer 54 side are not reflected at a boundary surface between the filler 80 and the backing material layer 54. It is preferable that the filler 80 is made of a member having heat radiation properties to increase an efficiency for radiating heat generated in the plurality of ultrasonic oscillators 48. In a case where the filler 80 has heat radiation properties, the filler 80 receives heat from the backing material layer 54, the substrate 60, the signal cable 110, and the like, and thus a heat radiation efficiency can be improved. A material of the filler 80 is not particularly limited, and for example, a silicone resin, rubber, or the like is used.

As shown in FIG. 7, the filler 80 fills a gap between an inner surface of the exterior member 41 and the first region AR1, the second region AR2, and the third region AR3, and is in contact with the first region AR1, the second region AR2, and the third region AR3.

With such a configuration, an anchor effect can be obtained by the filler 80 being embedded in a level difference at a boundary portion between the first region AR1 and the second region AR2, a level difference at a boundary portion between the second region AR2 and the third region AR3, or the like. As a result, a fixing force of various members by the filler 80 can be increased, and a durability of the ultrasonic endoscope 12 can be improved.

In addition, in a case where a smoothness of an outer surface of the second region AR2 is lower than a smoothness of an outer surface of the third region AR3, the filler 80 can also be embedded in an unevenness of the outer surface of the second region AR2 to obtain an anchor effect. As a result, the durability of the ultrasonic endoscope 12 can be further improved. In the present embodiment, the second region AR2 and the third region AR3 are disposed in the first space 410A, which is relatively narrow, in the accommodation space 410. Therefore, a volume of a gap between the exterior member 41 and the second region AR2 and the third region AR3 is small, and there is little room for the filler 80 to enter. Even in such a configuration, since the smoothness of the shield layer 108 is low, a sufficient fixing force can be secured even with a small amount of the filler 80.

As shown in FIGS. 7 and 8, a protrusion 102A that protrudes in a radial direction of the cable 100 is provided on an outer surface (surface of the outer cover 102) of a portion of the third region AR3 of the cable 100, which is disposed in the first space 410A.

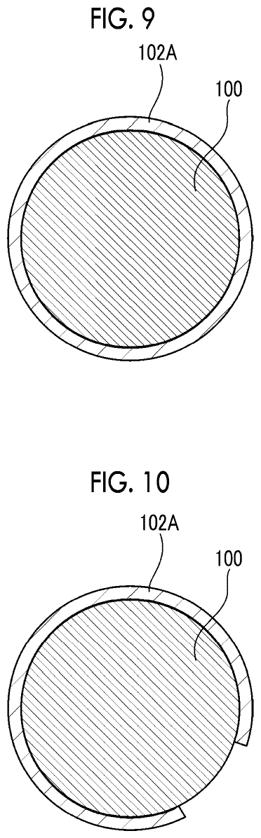

FIG. 9 is a schematic cross-sectional view taken along line A-A of FIG. 7. In FIG. 9, a cross section of the cable 100 is shown in a simplified manner. As shown in FIG. 9, the protrusion 102A is formed of an annular member provided on an entire circumference of an outer peripheral surface of the outer cover 102 of the cable 100 along the outer peripheral surface. An outer shape of the annular member is not particularly limited, and a true circle, an ellipse, a polygon, or the like can be adopted. The protrusion 102A may be integrally formed with the outer cover 102 of the cable 100, but is preferably a separate body from the cable 100. For example, by forming the protrusion 102A with a metal ring or the like, the cable 100 can be tightened by the protrusion 102A from an outer peripheral side thereof. As a result, it is possible to prevent the outer cover 102 from moving in an axial direction with respect to the shield layer 108 in the first space 410A. In addition, the filler 80 is embedded in the protrusion 102A so that an anchor effect can be obtained, and the durability of the ultrasonic endoscope 12 can be further improved.

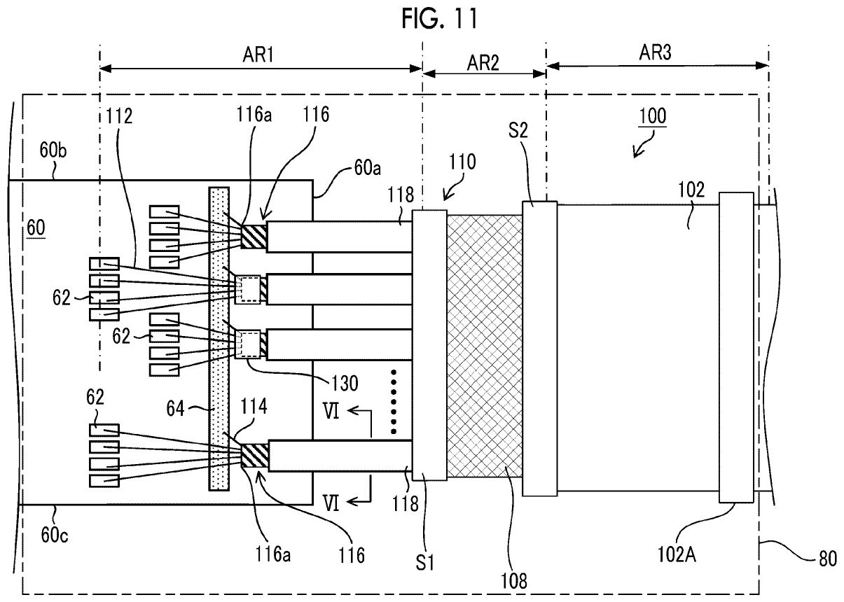

The protrusion 102A does not have to be provided on the entire circumference of the outer peripheral surface of the outer cover 102 of the cable 100 along the outer peripheral surface. For example, the protrusion 102A may have a C-shape as shown in FIG. 10. By forming the protrusion 102A in a C-shape, in a case where the cable 100 and the protrusion 102A are separate bodies, it is easy to mount the protrusion 102A on the cable 100. In addition, the anchor effect can be enhanced by the filler 80 being embedded between both circumferential ends of the C-shaped protrusion 102A. The C-shaped protrusion 102A shown in FIG. 10 is an example of an annular member.

In addition, as long as the purpose is to obtain the anchor effect, the protrusion 102A does not have to be formed of an annular member, and any shape can be adopted. By forming the protrusion 102A with an annular member, the anchor effect can be obtained while tightening the cable 100 as described above. A plurality of the protrusions 102A may be provided along an axial direction of the cable 100. As a result, the anchor effect can be further enhanced.

As shown in FIG. 7, the substrate 60 and the first signal line bundle 116 are fixed by a fixing part 130, and a relative position between the substrate 60 and each first signal line bundle 116 is fixed. The fixing part 130 fixes the substrate 60 and the first signal line bundle 116 in a state of overlapping the substrate 60. The first signal line bundle 116 composed of a twisted wire of the plurality of signal lines 112 and the plurality of ground lines 114 is disentangled into individual signal lines 112 at a distal end 116a. Each disentangled signal line 112 is electrically connected to the electrode pad 62 disposed on the substrate 60. The distal end 116a is a start position where each signal line 112 is disentangled. In addition, the fixing part 130 is omitted in some of the first signal line bundles 116 for ease of understanding. A connection region between the substrate 60 and the signal cable 110 as described above is also covered and fixed by the above-described filler 80.

In the ultrasonic oscillator unit 46 configured as described above, in a case where each ultrasonic oscillator 48 of the ultrasonic oscillator array 50 is driven, and a voltage is applied to the electrode 52 of the ultrasonic oscillator 48, the piezoelectric body 49 vibrates to sequentially generate ultrasonic waves, and the irradiation of the ultrasonic waves is performed toward the observation target site of the subject. Then, as the plurality of ultrasonic oscillators 48 are sequentially driven by an electronic switch, such as a multiplexer, scanning with ultrasonic waves is performed in a scanning range along a curved surface on which the ultrasonic oscillator array 50 is disposed, for example, a range of about several tens mm from a center of curvature of the curved surface.

In a case where the echo signal reflected from the observation target site is received, the piezoelectric body 49 vibrates to generate a voltage and outputs the voltage as an electric signal corresponding to the received ultrasound echo to the ultrasound processor device 14. The electric signal is subjected to various kinds of signal processing in the ultrasound processor device 14 and then is displayed as an ultrasound image on the monitor 20.

FIG. 11 is a view showing a modification example of the cable 100 and is a view corresponding to FIG. 7. The modification example shown in FIG. 11 is different from FIG. 7 in that a first sealing member S1 is provided at a first boundary portion between the first region AR1 and the second region AR2, and a second sealing member S2 is provided at a second boundary portion between the second region AR2 and the third region AR3.

The first sealing member S1 is provided to prevent the filler 80 from entering the inside of the shield layer 108. The second sealing member S2 is provided to prevent the filler 80 from entering between the outer peripheral surface of the shield layer 108 and an inner peripheral surface of the outer cover 102.

Materials of the first sealing member S1 and the second sealing member S2 are not particularly limited, and silicone-based resins, epoxy-based resins, or the like can be used. By providing the first sealing member S1 and the second sealing member S2, it is possible to prevent the filler 80 from entering the inside of the cable 100 deeply to the proximal end side in a case where the filler 80 before curing is poured into the accommodation space 410. As a result, even in a configuration in which the signal cable 110 has the second region AR2, flexibility of the cable 100 can be sufficiently secured on the proximal end side with respect to the distal end part 40.

It is preferable that a viscosity of a material constituting each of the first sealing member S1 and the second sealing member S2 is higher than a viscosity of a material constituting the filler 80. By increasing the viscosity of the materials of the first sealing member S1 and the second sealing member S2, in a case where the first boundary portion and the second boundary portion in the cable 100 are sealed by the first sealing member S1 and the second sealing member S2, it is possible to prevent the materials constituting the first sealing member S1 and the second sealing member S2 from entering the inside of the cable 100. The viscosity of the material constituting the filler 80 can be the same as or higher than the viscosity of the material constituting each of the first sealing member S1 and the second sealing member S2. However, in order to prevent fine air bubbles from being generated in the accommodation space 410, it is preferable that the viscosity of the material constituting the filler 80 is low. According to this modification example, since the first sealing member S1 and the second sealing member S2 are provided, even in a case where the filler 80 is formed using a material having a low viscosity, the filler 80 can be prevented from entering the inside of the cable 100. Therefore, the generation of air bubbles in the filler 80 can be suppressed, and heat radiation performance in the distal end part 40 can be improved.

In the modification example shown in FIG. 11, any one of the first sealing member S1 or the second sealing member S2 is not essential and may be omitted. Even in such a case, it is possible to obtain an effect of preventing the filler 80 from entering the inside of the cable 100, but a greater effect can be obtained by the presence of both the first sealing member S1 and the second sealing member S2.

In the above description, the filler 80 is in contact with the first region AR1, the second region AR2, and the third region AR3, but the present disclosure is not limited thereto. For example, the filler 80 may be configured to be in contact with the first region AR1 and the second region AR2 but not in contact with the third region AR3. Even in such a case, it is possible to improve a fixing force of various members by the filler 80 and to increase the durability of the ultrasonic endoscope 12.

In addition, the signal cable 110 is not limited to the non-coaxial cable as shown in FIG. 5, and may be a coaxial cable, a twisted pair cable, or the like. In a case where the signal cable 110 is a coaxial cable, for example, a configuration is adopted in which a shield layer is provided around one signal line 112, and the shield layer is covered with an insulating layer. In a case where the signal cable 110 is a twisted pair cable, a configuration is adopted in which two signal lines 112 are twisted together.

Although the ultrasonic endoscope 12 is a convex type, the technology of the present disclosure can also be applied to a radial type ultrasonic endoscope. In particular, in a configuration in which the ultrasonic observation part is provided on the distal end side with respect to the endoscope observation part in the radial type ultrasonic endoscope, there is a possibility that a cable connected to the ultrasonic observation portion is inserted into a narrow space of an exterior body of the distal end part. Therefore, the technology of the present disclosure is particularly effective.

As described above, at least the following matters are described in the present specification.

### 1

An ultrasonic endoscope comprising: a distal end part including an ultrasound transmission/reception section, in which the distal end part has a cable accommodation portion that accommodates a cable connected to the ultrasound transmission/reception section, and a filler that fills a gap in the cable accommodation portion, the cable has a plurality of signal cables that are electrically connected to ultrasonic oscillators included in the ultrasound transmission/reception section, a first covering member that bundles and covers the plurality of signal cables, and a second covering member that covers the first covering member, the cable includes, in order from an ultrasound transmission/reception section side, a first region in which the signal cable is exposed, a second region in which the first covering member is exposed, and a third region in which the second covering member is exposed, in the cable accommodation portion, and the filler is in contact with at least the first region and the second region.

### 2

The ultrasonic endoscope according to (1), in which the filler is further in contact with the third region.

### 3

The ultrasonic endoscope according to (2), in which a protrusion is provided on an outer surface of the third region.

### 4

The ultrasonic endoscope according to (3), in which the protrusion is formed of an annular member provided on the outer surface of the third region along a circumferential direction of the third region.

### 5

The ultrasonic endoscope according to any one of (1) to (4), in which an outer surface of the first covering member has a lower smoothness than an outer surface of the second covering member.

### 6

The ultrasonic endoscope according to (5), in which the first covering member exposed in the second region is a metal mesh shield.

### 7

The ultrasonic endoscope according to any one of (1) to (5), in which the cable includes a sealing member provided at at least one of a first boundary portion between the first region and the second region or a second boundary portion between the second region and the third region.

### 8

The ultrasonic endoscope according to (7), in which a viscosity of a material constituting the sealing member is higher than a viscosity of a material constituting the filler.

### 9

The ultrasonic endoscope according to any one of (1) to (8), in which the distal end part is provided with an imaging unit, and the ultrasound transmission/reception section is provided on a distal end side with respect to the imaging unit.

### Explanation of References

10: ultrasonic examination system
12: ultrasonic endoscope
14: ultrasound processor device
16: endoscope processor device
18: light source device
20: monitor
21a: water supply tank
21b: suction pump
22: insertion part
24: operating part
26: universal cord
28a: air/water supply button
28b: suction button
29: angle knob
30: treatment tool insertion port
32A, 32B, 32C: connector
34a: air/water supply tube
34b: suction tube
36: ultrasonic observation part
38: endoscope observation part
40: distal end part
41: exterior member
42: bendable part
43: soft part
44: treatment tool outlet port
45: treatment tool channel
46: ultrasonic oscillator unit
47: laminate
48: ultrasonic oscillator
49: piezoelectric body
50: ultrasonic oscillator array
52: electrode
52a: individual electrode
52b: oscillator ground
54: backing material layer
60: substrate
60a, 60b, 60c: side
62: electrode pad
64: ground electrode pad
76: acoustic matching layer
78: acoustic lens
80: filler
82: observation window
84: objective lens
86: imaging element
88: illumination window
90: cleaning nozzle
100: cable
101: covering portion
102: outer cover
102A: protrusion
106: resin layer
108: shield layer
110: signal cable
112: signal line
112a: conductor
112b: insulating layer
114: ground line
116: first signal line bundle
116a: distal end
118: sheath of first signal line bundle
130: fixing part
410: accommodation space
410A: first space
410B: second space
AR1: first region
AR2: second region
AR3: third region
S1: first sealing member
S2: second sealing member

What is claimed is:

1. An ultrasonic endoscope comprising:
a distal end part including an ultrasound transmission and reception section,
wherein the distal end part comprises a cable accommodation portion that accommodates a cable connected to the ultrasound transmission and reception section, and a filler that fills a gap in the cable accommodation portion,
the cable comprises a plurality of signal cables that are electrically connected to ultrasonic oscillators included in the ultrasound transmission and reception section, a first covering member that bundles and covers the plurality of signal cables, and a second covering member that covers the first covering member,
the cable has, in order from a side of the ultrasound transmission and reception section, a first region in which at least a part of the signal cables is exposed, a second region in which the first covering member is exposed, and a third region in which the second covering member is exposed, in the cable accommodation portion, and
the filler is in contact with at least the first region and the second region.

2. The ultrasonic endoscope according to claim 1,
wherein the filler is further in contact with the third region.

3. The ultrasonic endoscope according to claim 2,
wherein a protrusion is provided on an outer surface of the third region.

4. The ultrasonic endoscope according to claim 3,
wherein the protrusion is formed by an annular member provided on the outer surface of the third region along a circumferential direction of the third region.

5. The ultrasonic endoscope according to claim 1,
wherein an outer surface of the first covering member has a lower smoothness than an outer surface of the second covering member.

6. The ultrasonic endoscope according to claim 2,
wherein an outer surface of the first covering member has a lower smoothness than an outer surface of the second covering member.

7. The ultrasonic endoscope according to claim 3,
wherein an outer surface of the first covering member has a lower smoothness than an outer surface of the second covering member.

8. The ultrasonic endoscope according to claim 4,
wherein an outer surface of the first covering member has a lower smoothness than an outer surface of the second covering member.

9. The ultrasonic endoscope according to claim 5,
wherein the first covering member exposed in the second region is a metal mesh shield.

10. The ultrasonic endoscope according to claim 6,
wherein the first covering member exposed in the second region is a metal mesh shield.

11. The ultrasonic endoscope according to claim 7,
wherein the first covering member exposed in the second region is a metal mesh shield.

12. The ultrasonic endoscope according to claim 8,
wherein the first covering member exposed in the second region is a metal mesh shield.

13. The ultrasonic endoscope according to claim 1,
wherein the cable comprises a sealing member provided at at least one of a first boundary portion between the first region and the second region or a second boundary portion between the second region and the third region.

14. The ultrasonic endoscope according to claim 2,
wherein the cable comprises a sealing member provided at at least one of a first boundary portion between the first region and the second region or a second boundary portion between the second region and the third region.

15. The ultrasonic endoscope according to claim 3,
wherein the cable comprises a sealing member provided at at least one of a first boundary portion between the first region and the second region or a second boundary portion between the second region and the third region.

16. The ultrasonic endoscope according to claim 4,
wherein the cable comprises a sealing member provided at at least one of a first boundary portion between the first region and the second region or a second boundary portion between the second region and the third region.

17. The ultrasonic endoscope according to claim 13,
wherein a viscosity of a material constituting the sealing member is higher than a viscosity of a material constituting the filler.

18. The ultrasonic endoscope according to claim 1,
wherein the distal end part is provided with an imaging unit, and
the ultrasound transmission and reception section is provided on a distal end side with respect to the imaging unit.

19. The ultrasonic endoscope according to claim 2,
wherein the distal end part is provided with an imaging unit, and
the ultrasound transmission and reception section is provided on a distal end side with respect to the imaging unit.

20. The ultrasonic endoscope according to claim 3,
wherein the distal end part is provided with an imaging unit, and
the ultrasound transmission and reception section is provided on a distal end side with respect to the imaging unit.
